(19) United States
(12) Patent Application Publication
(10) Pub. No.: US 2013/0338525 A1
(43) Pub. Date: Dec. 19, 2013

(54) Mobile Human Interface Robot

(71) Applicant: iRobot Corporation, (US)

(72) Inventor: Thomas P. Allen, Reading, MA (US)

(73) Assignee: iRobot Corporation, Bedford, MA (US)

(21) Appl. No.: 13/869,280

(22) Filed: Apr. 24, 2013

Related U.S. Application Data
(60) Provisional application No. 61/637,757, filed on Apr. 24, 2012.

(51) Int. Cl. A61B 5/113; A61B 5/00; A61B 5/08

(52) U.S. Cl. CPC A61B 5/1135; A61B 5/08; A61B 5/746; A61B 5/0002
USPC 600/534

(57) ABSTRACT

A mobile robot that includes a drive system, a controller in communication with the drive system, and a volumetric point cloud imaging device supported above the drive system at a height of greater than about one foot above the ground. The volumetric point cloud imaging device monitors a plurality of translations of points in the point cloud corresponding to the surface of a respiratory center of a breathing subject. The controller receives point cloud signals from the imaging device and issues an alert command based at least in part on the received point cloud signals from the identified respiratory center.

### CROSS REFERENCE TO RELATED APPLICATIONS

This U.S. patent application claims priority under 35 U.S.C. §119(e) to U.S. Provisional Application Ser. No. 61/637,757, filed on Apr. 24, 2012. The disclosure of this prior application is considered part of the disclosure of this application and is hereby incorporated by reference in its entirety.

### TECHNICAL FIELD

The present invention relates to mobile human interface robots.

### BACKGROUND

A robot is generally an electro-mechanical machine guided by a computer or electronic programming. Mobile robots have the capability to move around in their environment and are not fixed to one physical location. An example of a mobile robot that is in common use today is an automated guided vehicle or automatic guided vehicle (AGV). An AGV is generally a mobile robot that follows markers or wires in the floor, or uses a vision system or lasers for navigation. Mobile robots can be found in industry, military and security environments. They also appear as consumer products, for entertainment or to perform certain tasks like vacuum cleaning and home assistance.

### SUMMARY

In one implementation, a mobile robot includes a drive system, a controller in communication with the drive system, and a volumetric point cloud imaging device supported above the drive system at a height of greater than about one foot above the ground. The volumetric point cloud imaging device monitors a plurality of translations of points in the point cloud corresponding to the surface of a respiratory center of a breathing subject. The controller receives point cloud signals from the imaging device and issues an alert command based at least in part on the received point cloud signals from the identified respiratory center. In some embodiments, issuing an alert command comprises communicating with the drive system and triggering autonomous relocation of the robot.

In some embodiments, the signals correspond to rate of movement and/or change in amplitude of the surface of the respiratory center of the breathing subject. In some embodiments, the alert command further comprises triggering an audible or visual alarm indicating an irregular respiratory condition corresponding to a rate of movement and/or change in amplitude waveform of the surface of the respiratory center of the breathing subject. In some embodiments, an alert condition may be identified including correlating the irregular change in conditions with a set of known conditions associated with one or more respiratory disorders.

In another implementation, a method of respiration detection for an autonomous mobile robot includes monitoring a plurality of translations of points in a volumetric point cloud, the monitored points corresponding to the surface of a respiratory center of a breathing subject. The method includes identifying an irregular change in the monitored plurality of translations, and issuing an alert command in response to the irregular change in the monitored plurality of translations.

In some embodiments, the method further includes applying a skeletal recognition algorithm that identifies a respiratory center of the subject based on the position and location of one or more skeletal components identified in the volumetric point cloud. In some embodiments, the irregular change in the monitored plurality of translations corresponds to a rate of movement and/or change in amplitude of the surface of the respiratory center of the breathing subject. In some embodiments, identifying an irregular change in the monitored plurality of translations further includes correlating the irregular change with a set of known conditions associated with respiratory disorders.

In some embodiments, issuing an alert command further comprises communicating with a robot controller. Issuing an alert command may further include triggering an audible or visual alarm on the robot indicative of an irregular respiratory condition corresponding to the translation of points. Issuing an alert command may include communicating with a drive system of the robot and triggering autonomous relocation of the robot.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1A is a perspective view of an exemplary mobile human interface robot having multiple sensors pointed toward the ground.

FIG. 1B is a perspective view of an exemplary mobile robot having multiple sensors pointed parallel with the ground.

FIG. 2 is a schematic view of an exemplary imaging sensor sensing an object in a scene.

FIG. 3 is a perspective view of an exemplary mobile human interface robot maintaining a sensor field of view on a person.

FIGS. 4A and 4B are perspective views of people interacting with an exemplary mobile human interface robot.

### DETAILED DESCRIPTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the expression “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

As used herein, the terms “center of respiration,” “respiration center” and “respiratory center” refer to a physical center of respiration of a subject. The respiratory center may be monitored and/or analyzed to determine a respiratory pattern. An exemplary respiratory center includes a chest and/or a torso of a subject.

Mobile robots can interact or interface with humans to provide a number of services that range from home assistance to commercial assistance and more. In the example of home assistance, a mobile robot can assist elderly people with everyday tasks, including, but not limited to, maintaining a medication regime, mobility assistance, communication assistance (e.g., video conferencing, telecommunications, Internet access, etc.), home or site monitoring (inside and/or outside), person monitoring, and/or providing a personal emergency response system (PERS). For commercial assistance, the mobile robot can provide videoconferencing (e.g., in a hospital setting), a point of sale terminal, interactive information/marketing terminal, etc.

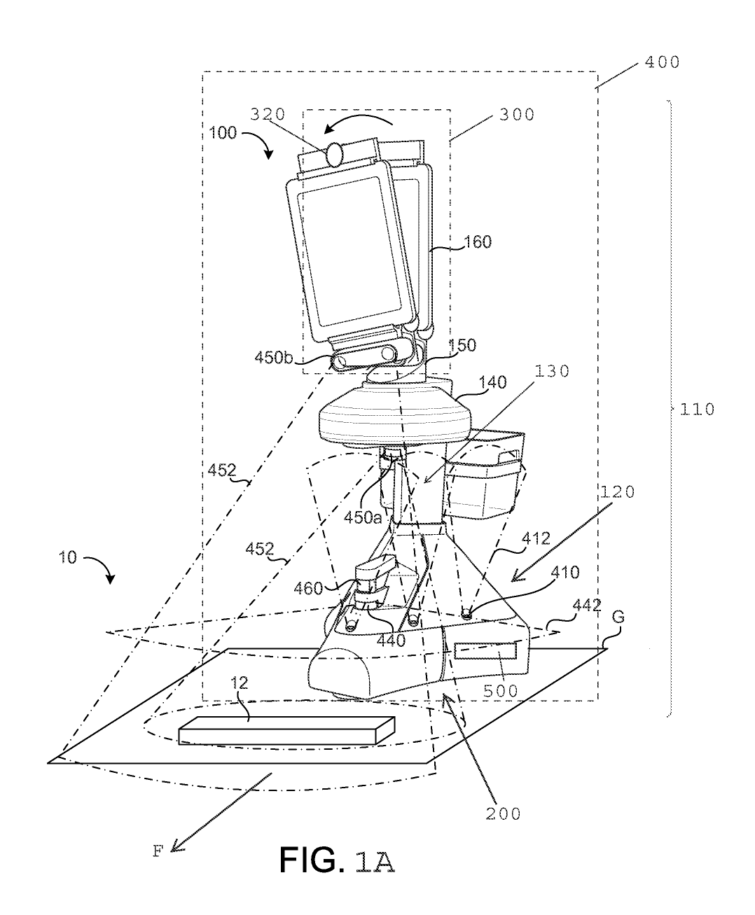

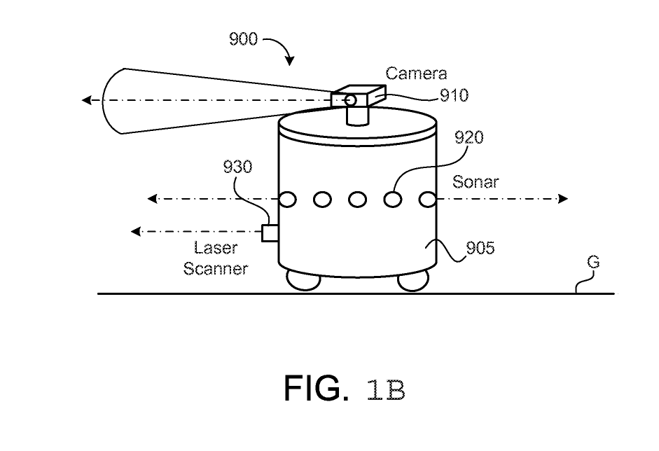

Referring to FIG. 1A, in some implementations, a mobile robot 100 includes a robot body 110 (or chassis) that defines a forward drive direction F. The robot 100 also includes a drive system 200, an interfacing module 300, and a sensor system 400, each supported by the robot body 110 and in communication with a controller 500 that coordinates operation and movement of the robot 100. A power source (e.g., battery or batteries) (not shown) can be carried by the robot body 110 and in electrical communication with, and deliver power to, each of these components, as necessary. For example, the controller 500 may include a computer capable of greater than 1000 MIPS (million instructions per second) and the power source provides a battery sufficient to power the computer for more than three hours.

In some implementations, the sensor system 400 includes a set or an array of proximity sensors 410 in communication with the controller 500 and arranged in one or more zones or portions of the robot 100 (e.g., disposed on or near the base body 120 of the robot body 110) for detecting any nearby or intruding obstacles. The proximity sensors 410 may be converging infrared (IR) emitter-sensor elements, sonar sensors, ultrasonic sensors, and/or imaging sensors (e.g., 3D depth map image sensors) that provide a signal to the controller 500 when an object is within a given range of the robot 100.

In some implementations, the sensor system 400 includes additional 3-D image sensors 450 disposed on the base body 120, the leg 130, the neck 150, and/or the head 160 of the robot body 110. In the example shown in FIG. 1A, the robot 100 includes 3-D image sensors 450 on the leg 130, the torso 140, and the neck 150. Other configurations are possible as well. One 3-D image sensor 450 (e.g., on the neck 150 and over the head 160) can be used for people recognition, gesture recognition, and/or videoconferencing, while another 3-D image sensor 450 (e.g., on the base 120 and/or the leg 130) can be used for navigation and/or obstacle detection and obstacle avoidance.

A forward facing 3-D image sensor 450 disposed on the neck 150 and/or the head 160 can be used for person, face, and/or gesture recognition of people about the robot 100. For example, using signal inputs from the 3-D image sensor 450 on the head 160, the controller 500 may recognize a user by creating a three-dimensional map of the viewed/captured user's face and comparing the created three-dimensional map with known 3-D images of people's faces and determining a match with one of the known 3-D facial images. Facial recognition may be used for validating users as allowable users of the robot 100. Moreover, one or more of the 3-D image sensors 450 can be used for determining gestures of person viewed by the robot 100, and optionally reacting based on the determined gesture(s) (e.g., hand pointing, waving, and or hand signals). For example, the controller 500 may issue a drive command in response to a recognized hand point in a particular direction or in response to in identifying a physical center (i.e., chest and/or torso) of respiration for monitoring and analyzing a respiratory pattern.

FIG. 1B provides a schematic view of a robot 900 having a camera 910, sonar sensors 920, and a laser range finder 930 all mounted on a robot body 905 and each having a field of view parallel or substantially parallel to the ground G. This arrangement allows detection of objects at a distance. In the example, a laser range finder 930 detects objects close to the ground G, a ring of ultrasonic sensors (sonars) 920 detect objects further above the ground G, and the camera 910 captures a large portion of the scene from a high vantage point. The key feature of this design is that the sensors 910, 920, 930 are all oriented parallel to the ground G. One advantage of this arrangement is that computation can be simplified, in the sense that a distance to an object determined by the using one or more of the sensors 910, 920, 930 is also the distance the robot 900 can travel before it contacts an object in a corresponding given direction.

In some implementations, the robot 100 includes a sonar scanner 460 for acoustic imaging of an area surrounding the robot 100. In the example shown in FIG. 1A, the sonar scanner 460 is disposed on a forward portion of the base body 120.

Referring to FIG. 1A, in some implementations, the robot 100 uses the laser scanner or laser range finder 440 for redundant sensing, as well as a rear-facing sonar proximity sensor 410 for safety, both of which are oriented parallel to the ground G. The robot 100 may include first and second 3-D image sensors 450a, 450b (depth cameras) to provide robust sensing of the environment around the robot 100. The first 3-D image sensor 450a is mounted on the torso 140 and pointed downward at a fixed angle to the ground G. By angling the first 3-D image sensor 450a downward, the robot 100 receives dense sensor coverage in an area immediately forward or adjacent to the robot 100, which is relevant for short-term travel of the robot 100 in the forward direction. The rear-facing sonar sensor 410 provides object detection when the robot travels backward. If backward travel is typical for the robot 100, the robot 100 may include a third 3D image sensor 450 facing downward and backward to provide dense sensor coverage in an area immediately rearward or adjacent to the robot 100.

The second 3-D image sensor 450b is mounted on the head 160, which can pan and tilt via the neck 150. The second 3-D image sensor 450b can be useful for remote driving since it allows a human operator to see where the robot 100 is going. The neck 150 enables the operator tilt and/or pan the second 3-D image sensor 450b to see both close and distant objects. Panning the second 3-D image sensor 450b increases an associated horizontal field of view. During fast travel, the robot 100 may tilt the second 3-D image sensor 450b downward slightly to increase a total or combined field of view of both 3-D image sensors 450a, 450b, and to give sufficient time for the robot 100 to avoid an obstacle (since higher speeds generally mean less time to react to obstacles). At slower speeds, the robot 100 may tilt the second 3-D image sensor 450b upward or substantially parallel to the ground G to track a person that the robot 100 is meant to follow. Moreover, while driving at relatively low speeds, the robot 100 can pan the second 3-D image sensor 450b to increase its field of view around the robot 100. The first 3-D image sensor 450a can stay fixed (e.g., not moved with respect to the base 120) when the robot is driving to expand the robot's perceptual range.

The 3-D image sensors 450 may be capable of producing the following types of data: (i) a depth map, (ii) a reflectivity based intensity image, and/or (iii) a regular intensity image. The 3-D image sensors 450 may obtain such data by image pattern matching, measuring the flight time and/or phase delay shift for light emitted from a source and reflected off of a target.

In some implementations, reasoning or control software, executable on a processor (e.g., of the robot controller 500), uses a combination of algorithms executed using various data types generated by the sensor system 400. The reasoning software processes the data collected from the sensor system 400 and outputs data for making navigational decisions on where the robot 100 can move without colliding with an obstacle, for example. By accumulating imaging data over time of the robot's surroundings, the reasoning software can in turn apply effective methods to selected segments of the sensed image(s) to improve depth measurements of the 3-D image sensors 450. This may include using appropriate temporal and spatial averaging techniques.

The reliability of executing robot collision free moves may be based on: (i) a confidence level built by high level reasoning over time and (ii) a depth-perceptive sensor that accumulates three major types of data for analysis—(a) a depth image, (b) an active illumination image and (c) an ambient illumination image. Algorithms cognizant of the different types of data can be executed on each of the images obtained by the depth-perceptive imaging sensor 450. The aggregate data may improve the confidence level a compared to a system using only one of the kinds of data.

The 3-D image sensors 450 may obtain images containing depth and brightness data from a scene about the robot 100 (e.g., a sensor view portion of a room or work area) that contains one or more objects. The controller 500 may be configured to determine occupancy data for the object based on the captured reflected light from the scene. Moreover, the controller 500, in some examples, issues a drive command to the drive system 200 based at least in part on the occupancy data to circumnavigate obstacles (i.e., the object in the scene). The 3-D image sensors 450 may repeatedly capture scene depth images for real-time decision making by the controller 500 to navigate the robot 100 about the scene without colliding into any objects in the scene. For example, the speed or frequency in which the depth image data is obtained by the 3-D image sensors 450 may be controlled by a shutter speed of the 3-D image sensors 450. In addition, the controller 500 may receive an event trigger (e.g., from another sensor component of the sensor system 400, such as proximity sensor 410, notifying the controller 500 of a nearby object or hazard. The controller 500, in response to the event trigger, can cause the 3-D image sensors 450 to increase a frequency at which depth images are captured and occupancy information is obtained.

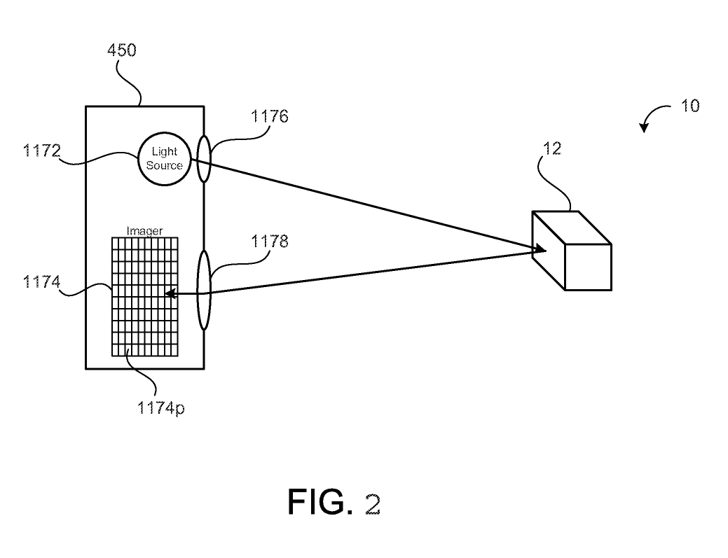

Referring to FIG. 2, in some implementations, the 3-D imaging sensor 450 includes a light source 1172 that emits light onto a scene 10, such as the area around the robot 100 (e.g., a room). The imaging sensor 450 may also include an imager 1174 (e.g., an array of light-sensitive pixels 1174p) which captures reflected light from the scene 10, including reflected light that originated from the light source 1172 (e.g., as a scene depth image). In some examples, the imaging sensor 450 includes a light source lens 1176 and/or a detector lens 1178 for manipulating (e.g., speckling or focusing) the emitted and received reflected light, respectively. The robot controller 500 or a sensor controller (not shown) in communication with the robot controller 500 receives light signals from the imager 1174 (e.g., the pixels 1174p) to determine depth information for an object 12 in the scene 10 based on image pattern matching and/or a time-of-flight characteristic of the reflected light captured by the imager 1174.

In some implementations, at least one of 3-D image sensors 450 can be a volumetric point cloud imaging device (such as a speckle or time-of-flight camera) positioned on the robot 100 at a height of greater than 1 or 2 feet above the ground and directed to be capable of obtaining a point cloud from a volume of space including a floor plane in a direction of movement of the robot (via the omni-directional drive system 200). In the examples shown in FIG. 1A, the first 3-D image sensor 450a can be positioned on the base 120 at height of greater than 1 or 2 feet above the ground (or at a height of about 1 or 2 feet above the ground) and aimed along the forward drive direction F to capture images (e.g., volumetric point cloud) of a volume including the floor while driving (e.g., for obstacle detection and obstacle avoidance). The second 3-D image sensor 450b is shown mounted on the head 160 (e.g., at a height greater than about 3 or 4 feet above the ground), so as to be capable of obtaining skeletal recognition and definition point clouds from a volume of space adjacent the robot 100. The controller 500 may execute skeletal/digital recognition software to analyze data of the captured volumetric point clouds. In some embodiments the first 3-D image sensor 450a and/or second 3-D image sensor 450b may be mounted to the robot via an articulated and/or telescoping arm for additional degrees of freedom and more particular orientation.

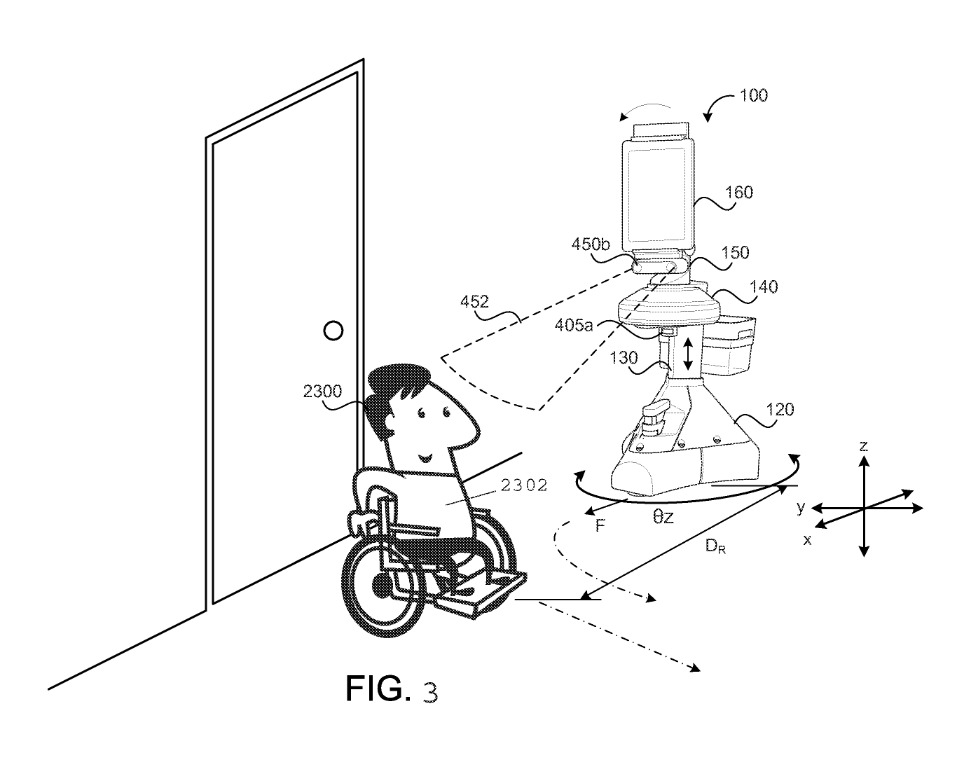

In some implementations, such as that shown in FIG. 3, a 3-D image sensor 450 is located at a height greater than 2 feet about the ground for alignment with a best skeletal location for monitoring respiration patterns and providing feeding feedback to the robot to initiate a response protocol. For example, the 3-D image sensor may sense joint angles and segment lengths to identify certain skeletal segments of a body, such as an arm and a head. Using the position and orientation of these segments and the relation(s) between them, a skeletal recognition algorithm can identify the location of a respiration center (i.e., chest 2302 and/or torso) of a breathing subject 2300 and instruct the robot 100 to align the 3-D sensor 450 with that respiration center to monitor respiration in the form of chest movement.

In other implementations, the 3-D sensor 450 may recognize a gesture, such as a hand tap to the respiratory center (i.e., a chest 2302 or torso) of a subject or an infrared laser pointer piloted remotely for localization upon the respiratory center. For example, a remote operator observing a subject via a drive camera co-occupying the same pan and tilt element as the 3-D sensor 450 may align a point or cross hair with the respiratory center 2302 and thereby direct the 3-D sensor 450 to emit upon the identified location.

Based on the identified respiratory center 2302, the robot 100 may respond by motoring around a subject to assume a best pose for monitoring respiration, which may be, for example, but not limited to, observing a prone subject from a vantage point aside the subject. In some implementations, a 3-D image sensor 450 can be a volumetric point cloud imaging device (such as a speckle or time-of-flight camera), and in other implementations a sonar sensor scans back and forth across the torso 2302 of a subject to detect respiration.

In some embodiments, the robot 100 runs an algorithm for identifying a respiratory center 2302 upon moving into view of a subject. In some embodiments, the robot runs the algorithm again following an external bump or displacement, thereby maintaining a best stationary pose and position for monitoring respiration of a subject. In some embodiments, the robot 100 runs the algorithm following movement of the subject, such as rolling onto a side. The robot 100 may reposition the 3-D sensor 450 in a best pose for monitoring respiration. For example, the 3-D sensor 450 may be mounted on an articulated and/or extendable and retractable arm (not shown) for positioning the 3-D sensor 450 above the subject and monitoring respiration from a side vantage point.

In some embodiments, the sensor 450 monitors a subject for learned respiratory conditions. The respiratory condition algorithm may be programmed initially with the variables and measurements associated with respiratory conditions such as sleep apnea, shallow breathing, asthma, etc. and, in some embodiments, the condition algorithm may learn and/or refine the variables and measurements associated with respiratory conditions. When the sensor 450 identifies a known set of conditions related to a respiratory abnormality or disorder, the robot 100 may respond to an alert command. In some embodiments, the robot 100 may respond to an alert command by making an alert sound audible to personnel and/or the robot 100 may transition from a stationary position to a mobile state to fetch personnel at a known location, such as nurse stationed at a known map location in a hospital ward. In some implementations, the alert command may trigger the robot 100 to display to the personnel the charted respiration data and summary statistics including, for example, respiration rate, amplitude, and identified issues such as those associated with the detected condition that triggered the alert condition response by the robot 100.

Additionally, in some implementations, the robot 100 issues a visible and/or audible alert which may be local to the robot 100 and/or remotely transmitted to a receiver monitored by personnel. In some implementations, the robot 100 is aware of the surroundings around a subject and moves from its vantage point to enable access to the subject by personnel advancing in response to the alert.

Incorporating a sensor, such as a 3-D sensor or distance ranger, on an autonomous mobile robotics platform for monitoring respiratory conditions provides many advantages. For example, a robot 100 having a respiratory condition monitoring algorithm may patrol a given ward autonomously (in a nursing home, hospital, orphanage, etc) during the night and passively monitor patients' sleep breathing, with no need for wired connection of a patient to a monitor. The robot 100 thereby removes the discomfort of patient constraints and frees hospital staff from the chore of manually checking respiration of a series of patients in routine rounds. Additionally, the robot 100 could report respiratory data and identified conditions directly into hospital EMR systems. Doctors performing remote telemedicine consultations through the robot 100 could respond independently to readings without requiring interaction with local personnel. In other uses, for example, the respiratory condition monitoring algorithm mounted to a mobile platform provides feedback for breathing coaching as part of robot-assisted after care, rehabilitation, etc. The robot 100, while coaching a person through exercises, autonomously monitors the subject's breathing and ensures compliance with instructions (e.g., instructions to take deeper breaths, to concentrate on measured breathing, etc.).

In some embodiments, a controller 500 may use imaging data from the imaging sensor 450 for color/size/dimension blob matching. Identification of discrete objects 12 in the scene 10 (FIG. 1A) allows the robot 100 to not only avoid collisions, but also to search for objects 12. The human interface robot 100 may need to identify humans and target objects 12 against the background of a home or office environment. The controller 500 may execute one or more color map blob-finding algorithms on the depth map(s) derived from the imaging data of the imaging sensor 450 as if the maps were simple grayscale maps and search for the same “color” (that is, continuity in depth) to yield continuous objects 12 in the scene 10. Using color maps to augment the decision of how to segment objects 12 would further amplify object matching, by allowing segmentation in the color space as well as in the depth space. The controller 500 may first detect objects 12 by depth, and then further segment the objects 12 by color. This allows the robot 100 to distinguish between two objects 12 close to or resting against one another with differing optical qualities. Color/size/dimension blob matching may be used to identify a subjects' respiratory center. For example, the imaging sensor 450 using skeletal and/or gesture recognition may detect the presence and orientation of a hand contrasted against a blanket and a head contrasted against a pillow, thereby enabling the robot 100 to determine a relative position of a chest and/or torso.

‘Dense data’ vs. ‘sparse data’ and ‘dense features’ vs. ‘sparse features’ are referred to herein with respect to spatial data sets. Without limiting or narrowing the meaning from that of those skill in the art would interpret such terms to mean, ‘dense’ vs. ‘sparse’ generally means many data points per spatial representation vs. few data points, and specifically may mean: (i) in the context of 2-D image data or 3-D ‘images’ including 2-D data and range, ‘dense’ image data includes image data substantially fully populated with pixels, or capable of being rasterized to pixels with substantially no losses and/or artifacting from the original image capture (including substantially uncompressed, raw, or losslessly compressed images), while a ‘sparse’ image is one where the image is quantized, sampled, lossy compressed, vectorized, segmented (e.g., into superpixels, nodes, edges, surfaces, interest points, voxels), or otherwise materially reduced in fidelity from the original capture, or must be interpolated in being rasterized to pixels to re-represent an image; (ii) in the context of 2-D or 3-D features, ‘dense features’ may be features that are populated in a substantially unconstrained manner, to the resolution of the detection approach - all that can be detected and recorded, and/or features that are recognized by detectors recognized to collect many features (HOG, wavelets) over a sub-image; ‘sparse features’ may be purposefully constrained in number, in the number of feature inputs, lateral inhibition, and/or feature selection, and/or may be recognized by detectors recognized to identify a limited number of isolated points in an image (e.g., Harris corner, edges, Shi-Tomasi).

With respect to 3-D environment structure, the robot may acquire images, such as dense images, of a scene including a patient of interest (e.g., a respiration monitoring target). In some implementations, the robot uses a camera and/or an imaging sensor (e.g., volumetric point cloud imaging device) for obtaining the dense images. The controller, which is in communication with the camera and/or the imaging sensor may associate information with the dense images (e.g., annotate or tag the dense images with data), such as patient identity information, other respiration or health sensed concurrently with respiration information by another device (e.g., blood pressure sensed by a blood pressure monitor, oxygen level sensed by a pulse oximeter, blood glucose sensed by a blood glucose meter, respiratory peak flow sensed by a peak flow meter), along with timestamps. The image data may be raw sensor data (e.g., a point cloud or signal or the dense image sequence).

After a threshold period of time or a threshold amount of image data , the robot or a cloud service may execute one of a variety of on-line or off-line methods to process the image data set into a dense 3-D map or model of the scene (environment) and then simplify this dense 3-D map or model into a 2-D height map of a respiratory patient's chest 2302, which can also include a 2-D—map of differential height data at each point (e.g., a 2-D topographical map of a user's chest 2302 as well as a 2-D map of change in respiratory displacement, “2-D+min/max”). In some examples, the 2-D height map is a topographical map having X and Y coordinates with Z data. Each X,Y coordinate may have one or more Z points (i.e., height data). Unlike the dense 3-D map, which may have numerous Z points (e.g., hundreds or thousands of Z points) for each X,Y coordinate, the 2-D height map may have less than threshold number of Z points for each X,Y coordinate, such as between two and twenty (e.g., ten) points. A 2-D height map derived from a 3-D map of a respirating patient may show a first Z point for the “bottom dead center” of each point on the patient's chest during respiration and a second Z point for the “top dead center” of the same points. This information can map total displacement or various patterns of respiration. By reducing the Z-points from a dense data set of a continuous range of Z points for each X, Y coordinate to a sparse data set of a select number of Z points indicative of the position and movement of the user's chest representative of the chest cavity, the robot can receive a 2-D height map having a relatively smaller size than the 3-D map. This, in turn, allows the robot to store the 2-D height map on local memory having a practical and cost effective size.

The robot or off-line cloud service may execute one or more filters (e.g., a Bundle Adjustment, RANSAC, Expectation Maximization, SAM or other 3-D structural estimation algorithms) for processing an image data set into a 3-D representation.

With respect to respiratory pattern classification, the robot may acquire images of the respiratory patient's chest 2302 (FIG. 3). Once the annotated image data sets are accumulated (potentially along with the data set from many other robots), a parallel cloud host may be launched to process the annotated image data set using a supervised learning algorithm, for example, that computes respiratory diagnostic or respiratory incident classes from the many images of real patients' chests 2302. Once the training of a diagnostic image class model is complete, the parameters for that model (a small amount of data) can be downloaded back down to many different robots. Learning methods applicable to this method include genetic algorithms, neural networks, and support vector machines. All of these may be too complex and may take too much storage to run on-line (i.e., on a local robot processor) in a low-cost robot, but a cloud offers a robot fleet access to “fully trained” classifiers.

Referring again to FIG. IA, the first and second 3-D image sensors 450a, 450b can be used to improve mapping of the robot's environment to create a robot map, as the first 3-D image sensor 450a can be used to map out nearby objects and the second 3-D image sensor 450b can be used to map out distant objects.

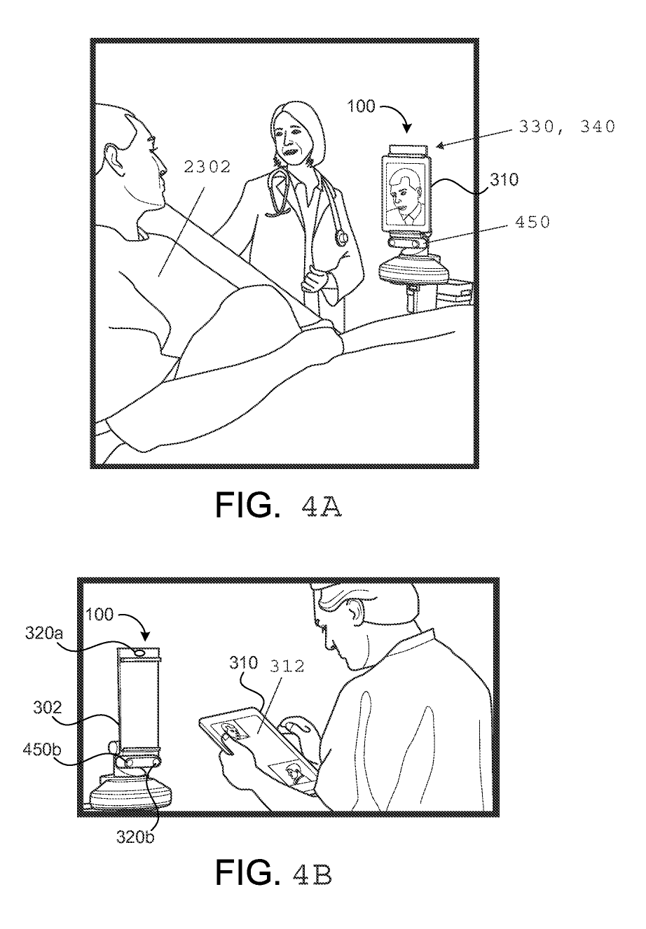

Referring to FIGS. 3 and 4A, in some implementations, the robot 100 may detect, track, and follow a person 2300. Since the robot 100 can pan and tilt the head 160 using the neck 150, the robot 100 can orient the second 3-D image sensor 450b to maintain a corresponding field of view 452 on the person 2300, and in particular on the chest 2302 of the person 2300. Moreover, since the head 160 can move relatively more quickly than the base 120 (e.g., using the drive system 200), the head 160 (and the associated second 3-D image sensor 450b) can track the person 2300 more quickly than by turning the robot 100 in place. The robot 100 can drive toward the person 2300 to keep the person 2300 within a threshold distance range D_{R} (e.g., corresponding to a sensor field of view). In some examples, the robot 100 turns to face forward toward the person/user 2300 while tracking the person 2300. The robot 100 may use velocity commands and/or waypoint commands to follow the person 2300.

With reference to FIGS. 1A, 4A and 4B, in some implementations, the head 160 supports one or more portions of the interfacing module 300. The head 160 may include a dock 302 for releasably receiving one or more computing tablets 310, also referred to as a web pad or a tablet PC, each of which may have a touch screen 312. The web pad 310 may be oriented forward, rearward or upward. In some implementations, web pad 310 includes a touch screen, optional I/O (e.g., buttons and/or connectors, such as micro-USB, etc.) a processor, and memory in communication with the processor. An exemplary web pad 310 is the Apple iPad by Apple, Inc. In some examples, the web pad 310 functions as the controller 500 or assist the controller 500 and controlling the robot 100.

In some implementations, the robot 100 includes multiple web pad docks 302 on one or more portions of the robot body 110. For example, the robot 100 may include a web pad dock 302 optionally disposed on the leg 130 and/or the torso 140. This allows the user to dock a web pad 310 at different heights on the robot 100, for example, to accommodate users of different height, capture video using a camera of the web pad 310 in different vantage points, and/or to receive multiple web pads 310 on the robot 100.

The interfacing module 300 may include a camera 320 disposed on the head 160 which can be used to capture video from elevated vantage point of the head 160 (e.g., for videoconferencing). In the example shown in FIG. 4B, the camera 320b is disposed on the neck 150. In some examples, the camera 320 is operated only when the web pad 310 is detached or undocked from the head 160. When the web pad 310 is attached or docked on the head 160 in the dock 302 (and optionally covering the camera 320), the robot 100 may use a camera of the web pad 310 for capturing video. In such instances, the camera 320 may be disposed behind the docked web pad 310 and enters an active state when the web pad 310 is detached or undocked from the head 160 and an inactive state when the web pad 310 is attached or docked on the head 160.

The robot 100 can provide videoconferencing (e.g., at 24 fps) through the interface module 300 (e.g., using a web pad 310, the camera 320, microphone(s) 330, and/or speaker(s) 340). The videoconferencing can be multiparty. The robot 100 can provide eye contact between both parties of the videoconferencing by maneuvering the head 160 to face the user. Moreover, the robot 100 can have a gaze angle of less than 5 degrees (e.g., an angle away from an axis normal to the forward face of the head 160). At least one 3-D image sensor 450 and/or the camera 320 on the robot 100 can capture life size images including body language. The controller 500 can synchronize audio and video (e.g., with the difference of less than 50 ms). In the embodiments shown in FIGS. 4A and 4B, the robot 100 can provide videoconferencing for people standing or sitting by adjusting the height of the web pad 310 on the head 160 and/or the camera 320 (by raising or lowering the torso 140) and/or panning and/or tilting the head 160. The camera 320 may be movable within at least one degree of freedom separately from the web pad 310. In some embodiments, the camera 320 has an objective lens positioned more than 3 feet from the ground, but no more than 10 percent of the web pad height from a top edge of a display area of the web pad 310. Moreover, the robot 100 can zoom the camera 320 to obtain close-up pictures or video about the robot 100. The head 160 may include one or more speakers 340 so as to have sound emanate from the head 160 near the web pad 310 displaying the videoconferencing.

In some embodiments, the robot 100 can receive user inputs into the web pad 310 (e.g., via a touch screen 312), as shown in FIG. 4B. In some implementations, the web pad 310 is a display or monitor, while in other implementations the web pad 310 is a tablet computer. The web pad 310 can have easy and intuitive controls, such as a touch screen, providing high interactivity. The web pad 310 may have a monitor display 312 (e.g., touch screen) having a display area of 150 square inches or greater and may be movable with at least one degree of freedom.

In the example shown in FIG. 4B, a user may remove the web pad 310 from the web pad dock 302 on the head 160 for remote operation of the robot 100, videoconferencing (e.g., using a camera and microphone of the web pad 310), and/or usage of software applications on the web pad 310. The robot 100 may include first and second cameras 320a, 320b on the head 160 to obtain different vantage points for videoconferencing, navigation, etc., while the web pad 310 is detached from the web pad dock 302.

Interactive applications executable on the controller 500 and/or device(s) in communication with the controller 500 may require more than one display on the robot 100. Multiple web pads 310 associated with the robot 100 can provide different combinations of “FaceTime”, Telestration, HD look at this-cam (e.g., for web pads 310 having built in cameras), can act as a remote operator control unit (OCU) for controlling the robot 100 remotely, and/or can provide a focal user interface pad.

The operations described herein as being carried out or executed by the controller 500 and/or device(s) in communication with the controller 500 may be programmatically carried out or executed by the controller 500 and/or the device(s) in communication with the controller 500. The term “programmatically” refers to operations directed and/or primarily carried out electronically by computer program modules, code and instructions.

While this specification contains many specifics, these should not be construed as limitations on the scope of the invention or of what may be claimed, but rather as descriptions of features specific to particular implementations of the invention. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

What is claimed is:

1. A mobile robot comprising:
a drive system;
a controller in communication with the drive system; and
a volumetric point cloud imaging device supported above the drive system at a height of greater than about one foot above the ground, the imaging device configured to monitor a plurality of translations of points in a point cloud corresponding to a surface of a respiratory center of a breathing subject;
wherein the controller is configured to receive point cloud signals from the imaging device and issue an alert command based at least in part on the received point cloud signals from the respiratory center.

2. The mobile robot of claim 1, wherein the signals correspond to rate of movement and change in amplitude of the surface of the respiratory center of the breathing subject.

3. The mobile robot of claim 1, wherein the alert command comprises a triggered audible or visual alarm indicating an irregular respiratory condition corresponding to a rate of movement and/or change in amplitude of the surface of the respiratory center of the breathing subject.

4. The mobile robot of claim 1, wherein the controller is configured to issue an alert command including communicating with the drive system and triggering autonomous relocation of the robot.

5. The mobile robot of claim 1, wherein the controller is configured to identify an alert condition including correlating an irregular change in conditions with a set of known conditions associated with one or more respiratory disorders.

6. A method of respiration detection for an autonomous mobile robot, the method comprising, using the robot:
monitoring a plurality of translations of points in a volumetric point cloud, the monitored points corresponding to a surface of a respiratory center of a breathing subject;
identifying an irregular change in the monitored plurality of translations; and
issuing an alert command in response to the irregular change in the monitored plurality of translations.

7. The method of claim 6, further comprising applying a skeletal recognition algorithm that identifies a respiratory center of the subject based on the position and location of one or more skeletal components identified in the volumetric point cloud.

8. The method of claim 6, wherein the irregular change in the monitored plurality of translations corresponds to a rate of movement and/or change in amplitude of the surface of the respiratory center of the breathing subject.

9. The method of claim 6, wherein identifying the irregular change in the monitored plurality of translations further comprises correlating the irregular change with a set of known conditions associated with respiratory disorders.

10. The method of claim 6, wherein issuing an alert command further comprises communicating with a robot controller.

11. The method of claim 10, wherein issuing an alert command further comprises triggering an audible or visual alarm on the robot indicative of an irregular respiratory condition corresponding to the translation of points.

12. The mobile robot of claim 10, wherein issuing an alert command comprises communicating with a drive system of the robot and triggering autonomous relocation of the robot.
